(19) United States
(12) Patent Application Publication
(10) Pub. No.: US 2015/0133790 A1
(43) Pub. Date: May 14, 2015

(54) GAIT ANALYSIS DEVICE AND RUNNING EXERCISE APPARATUS HAVING THE SAME

(71) Applicant: NATIONAL CHENG KUNG UNIVERSITY, Tainan City (TW)

(72) Inventors: Hung-Ta CHIU, Tainan City (TW); Lih-Yih CHIOU, Tainan City (TW); Ming-You HSU, Kaohsiung City (TW); Pei-Wei JI, Taichung City (TW)

(73) Assignee: NATIONAL CHENG KUNG UNIVERSITY, Tainan City (TW)

(21) Appl. No.: 14/199,429

(22) Filed: Mar. 6, 2014

(30) Foreign Application Priority Data
Nov. 12, 2013 (TW) 102141144

(51) Int. Cl. A61B 5/11; A61B 5/00

(52) U.S. Cl. CPC A61B 5/112; A61B 5/0059; A61B 5/1126
USPC 600/473

(57) ABSTRACT

A gait analysis device cooperated with a running exercise apparatus comprises a plurality of infrared emitting units, a plurality of infrared receiving units, a plurality of supporting units and a processing unit. An end of each of the infrared emitting units is covered by an opaque element. The infrared receiving units and the infrared emitting units are disposed in a staggered arrangement. The infrared receiving units and the infrared emitting units are disposed to the supporting units. The processing unit is coupled with the infrared receiving units. A running exercise apparatus is also disclosed. The gait analysis device is capable of decreasing the interference of the infrared light and thus increasing the accuracy of the gait analysis.

### CROSS REFERENCE TO RELATED APPLICATIONS

This Non-provisional application claims priority under 35 U.S.C. §119(a) on Patent Application No(s). 102141144 filed in Taiwan, Republic of China on Nov. 12, 2013, the entire contents of which are hereby incorporated by reference.

### BACKGROUND OF THE INVENTION

1. Field of Invention

The invention relates to a gait analysis device and a running exercise apparatus having the same and, in particular, to a gait analysis device and a running exercise apparatus having the same which can decrease the interference.

2. Related Art

With the more importance on human health, various running exercise apparatuses are developed nowadays, and a treadmill is a basic and common apparatus for the user's exercise to keep the fitness and release the user from the life pressure. Besides, with the progress of technologies, the treadmill has been further applied to the training of a professional athlete or even to the medical rehabilitation purpose. Hence, to achieve the above-mentioned purposes, the device disposed on the treadmill for acquiring the exercise information has become the mainstream of the field of the running exercise apparatus.

The current running exercise apparatus for the exercise purpose or the rehabilitation purpose is equipped with the device that can analyze the gait information of the user, and the user can know the exercise condition by the acquired gait information such as stride frequency. It is better for the medical rehabilitation that the rehabilitation condition of the patient can be made clearer thereby, and the rehabilitation effect can be thus enhanced. The said devices for acquiring the information include an emitter or a reflective mark that is disposed on the user's body such as the foot, and include a corresponding receiver disposed on the running exercise apparatus to receive the signal emitted by the device worn by the user. Otherwise, a plurality of detecting devices including emitters and receivers are disposed on the left and right sides of the running exercise apparatus, respectively, and the exercise condition of the user can be detected by intercepting the light through the emitter and the receiver by the user's feet.

However, for the former method mentioned above, the user needs to wear the devices and the devices thus become the additional burden for the user on the exercise or rehabilitation and disadvantage the usage. For the latter method mentioned above, although the user needn't wear any device, it is necessary to dispose the devices with a higher density for acquiring more accurate information of the user's exercise. Besides, the detecting devices for acquiring the user's gait information mostly use the infrared transmission and reception technology. However, when the adjacent infrared transmitters are disposed too closely, the light beams emitted by them may interfere with each other and therefore the signal accuracy is decreased and the error will be added to the analysis result.

Therefore, it is an important subject to provide a gait analysis device applied to a running exercise apparatus that can avoid the interference during the detection so as to acquire the more accurate gait information of the user without adding an additional burden to the user.

### SUMMARY OF THE INVENTION

In view of the foregoing subject, an objective of the invention is to provide a gait analysis device applied to a running exercise apparatus that can avoid the interference during the detection so as to acquire the more accurate gait information of the user without adding an additional burden to the user.

To achieve the above objective, a gait analysis device according to the invention is cooperated with a running exercise apparatus and comprises a plurality of infrared emitting units, a plurality of infrared receiving units, a plurality of supporting units and a processing unit. An end of each of the infrared emitting units is covered by an opaque element. The infrared receiving units and the infrared emitting units are disposed in a staggered arrangement. The infrared receiving units and the infrared emitting units are disposed to the supporting units. The processing unit is coupled with the infrared receiving units.

In one embodiment, each of the infrared emitting units, each of the infrared receiving units or each of the supporting units has a bent angle corresponding to at least an edge of the running exercise apparatus.

In one embodiment, the gait analysis device further comprises a connecting unit connected to the supporting units.

In one embodiment, the infrared emitting units are modulated IR emitting units and the infrared receiving units are modulated IR receiving units.

In one embodiment, one of the infrared emitting units and the infrared receiving unit adjacent to the infrared emitting unit have an interval of 3 cm˜5 cm.

In one embodiment, the distance between one of the infrared emitting units and the infrared receiving unit that is opposite to the infrared emitting unit is 55 cm˜80 cm.

In one embodiment, the gait analysis device further comprises an accelerometer coupled to the processing unit.

To achieve the above objective, a running exercise apparatus according to the invention comprises a main body and a gait analysis device. The gait analysis device is disposed corresponding to the main body and comprises a plurality of infrared emitting units, a plurality of infrared receiving units, a plurality of supporting units and a processing unit. An end of each of the infrared emitting units is covered by an opaque element. The infrared receiving units and the infrared emitting units are disposed in a staggered arrangement. The infrared receiving units and the infrared emitting units are disposed to the supporting units. The processing unit is coupled with the infrared receiving units.

In one embodiment, each of the infrared emitting units, each of the infrared receiving units or each of the supporting units has a bent angle corresponding to at least an edge of the main body.

In one embodiment, the gait analysis device further comprises a connecting unit connected to the supporting units.

In one embodiment, the infrared emitting units are modulated IR emitting units and the infrared receiving units are modulated IR receiving units.

In one embodiment, one of the infrared emitting units and the infrared receiving unit adjacent to the infrared emitting unit have an interval of 3 cm˜5 cm.

In one embodiment, the distance between one of the infrared emitting units and the infrared receiving unit that is opposite to the infrared emitting unit is 55 cm˜80 cm.

In one embodiment, the gait analysis device further comprises an accelerometer coupled to the processing unit.

As mentioned above, the gait analysis device and the running exercise apparatus according to the invention have the infrared modules disposed on two sides of the running exercise apparatus, and the infrared emitting units and the infrared receiving units of the adjacent infrared modules are disposed in a staggered arrangement. Accordingly, the interval between the adjacent infrared receiving units can be increased and the interference therebetween can be thus effectively decreased. Besides, the emission end of the infrared emitting unit can be covered by an opaque element. The opaque element reduces the scattering of the infrared light emitted by the infrared emitting unit, and therefore the accuracy of the light signal obtained by the gait analysis device and the correctness of the analysis result can be both enhanced. Furthermore, the user can know the gait information of the motion thereby.

In order to avoid the interference between the infrared modules, the running exercise apparatus of the conventional art needs to use the modulated IR signal technique that requires the higher cost and higher level technology. However, in the invention, since the units are disposed in a staggered arrangement and the infrared emitting unit is covered by an opaque element, the gait analysis device can use the unmodulated IR signal technique but still reduce the interference caused by the unmodulated IR signal technique. Therefore, the invention is capable of reducing the cost of the running exercise apparatus.

### BRIEF DESCRIPTION OF THE DRAWINGS

The invention will become more fully understood from the detailed description and accompanying drawings, which are given for illustration only, and thus are not limitative of the present invention, and wherein:

FIG. 1 is a schematic diagram of a running exercise apparatus including a gait analysis device according to an embodiment of the invention;

FIG. 2 is a schematic top-view diagram of a part of the running exercise apparatus in FIG. 1;

FIG. 3 is a schematic diagram of a part of the gait analysis device of the running exercise apparatus in FIG. 1;

FIG. 4 is a schematic enlarged diagram of a part of the running exercise apparatus in FIG. 1;

FIG. 5 is a schematic block diagram of the running exercise apparatus in FIG. 1;

FIG. 6A is a schematic diagram showing a foot state when the gait analysis is implemented by the running exercise apparatus in FIG. 1;

FIG. 6B is a schematic diagram showing the detection result of the foot state in FIG. 6A;

FIG. 7A is a schematic diagram showing another foot state when the gait analysis is implemented by the running exercise apparatus in FIG. 1; and

FIG. 7B is a schematic diagram showing the detection result of the foot state in FIG. 7A.

### DETAILED DESCRIPTION OF THE INVENTION

The present invention will be apparent from the following detailed description, which proceeds with reference to the accompanying drawings, wherein the same references relate to the same elements.

To be noted, the running exercise apparatus of the invention can be applied to the user's gait analysis, which is capable of systematically measuring, describing and estimating the quantified parameters of the human motion ability for example. By the gait analysis, the space-time parameters, kinematics and dynamics related to the user's foot can be calculated and the basic properties of the gait can be analyzed, and therefore the incorrect gait of the user can be corrected or the medical reference for the clinical or rehabilitated patient can be provided.

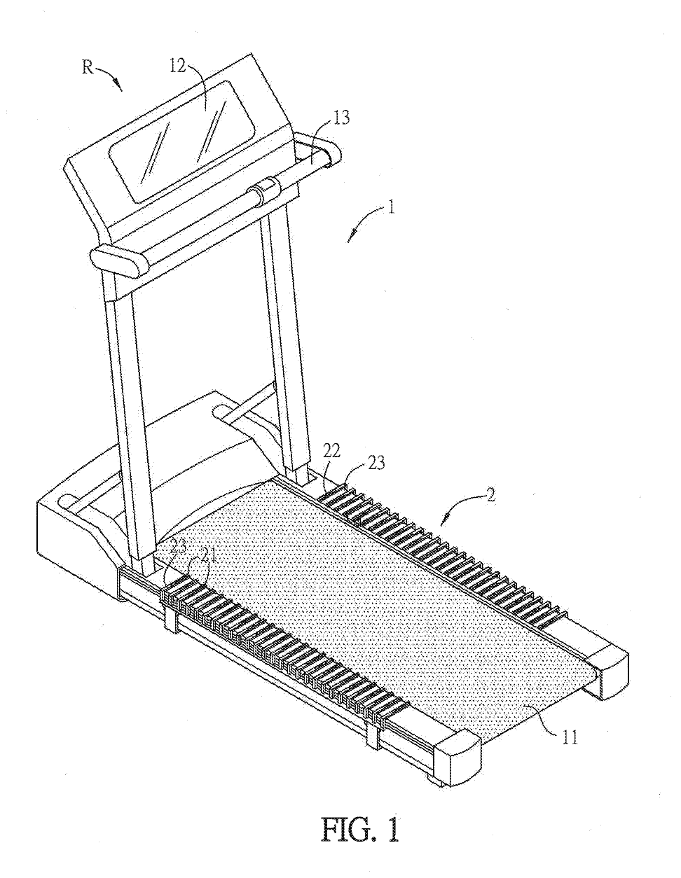

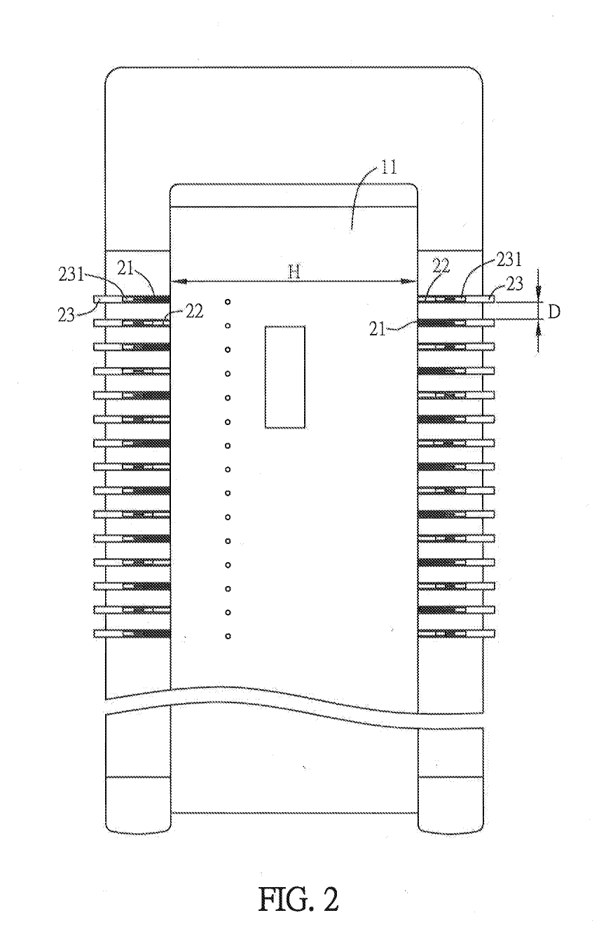

FIG. 1 is a schematic diagram of a running exercise apparatus including a gait analysis device according to an embodiment of the invention, and FIG. 2 is a schematic top-view diagram of a part of the running exercise apparatus in FIG. 1. As shown in FIGS. 1 and 2, the running exercise apparatus R includes a main body 1 and a gait analysis device 2, and the gait analysis device 2 is disposed corresponding to the main body 1. To be noted, the main body 1 of the running exercise apparatus R of this embodiment is a treadmill for the exercise, training or rehabilitation for example. The main body 1 may include the same components as a normal treadmill, the components such as a rolling portion 11, a display portion 12 for displaying information and a hold portion for being held by the user. In this embodiment, the rolling portion 11 is a belt-like structure and can be rolled by a motor (not shown) for example to cause the movement of the user's feet. The other structures and the related operation mechanism of the main body 1 can be comprehended by those skilled in the art, and therefore they are not described here for conciseness. The following is the illustration about the structure and operation mechanism of the gait analysis device 2.

The gait analysis device 2 is disposed at the edges of the main body 1, and for securing the safety of the user, the gait analysis device 2 doesn't contact the rolling portion 11 substantially. In this embodiment, the gait analysis device 2 includes a plurality of infrared modules and a plurality of supporting units 23. The infrared module is composed of an infrared emitting unit 21 and an infrared receiving unit 22. Each of the infrared emitting unit 21 and the infrared receiving unit 22 is disposed to a supporting unit 23. Because the supporting units 23 are disposed at the edges of the main body 1, the infrared emitting units 21 and the infrared receiving units 22 can be disposed on the opposite sides of the rolling portion 11.

As an embodiment, the supporting unit 23 includes a recess 231 for example, and the infrared emitting units 21 and the infrared receiving units 22 can be stably disposed in the recesses 231. In this case, other fixing means, such as an adhesive, is not required. However, the invention is not limited thereto. In other embodiments, the supporting units 23 can include other structures for fixing the infrared emitting units 21 and the infrared receiving units 22, or the adhesive can be used to fix the above-mentioned units.

In this embodiment, the supporting unit 23 is bent with an angle, corresponding to at least an edge of the main body 1. As an embodiment, the supporting unit 23 is an L-shaped structure with a bent angle near 90 degrees so that it can be attached to the edge of the main body 1 to provide a fixing effect. In other embodiments, the infrared emitting unit 21 or the infrared receiving unit 22 also can have a bent angle corresponding to at least an edge of the main body 1 for obtaining a better fixing effect. However, the shapes of the above-mentioned infrared emitting units 21, infrared receiving units 22 and supporting units are just for the illustration but not for limiting the scope of the invention, and they can be cooperated with one another as long as they can be stably fixed to the main body 1.

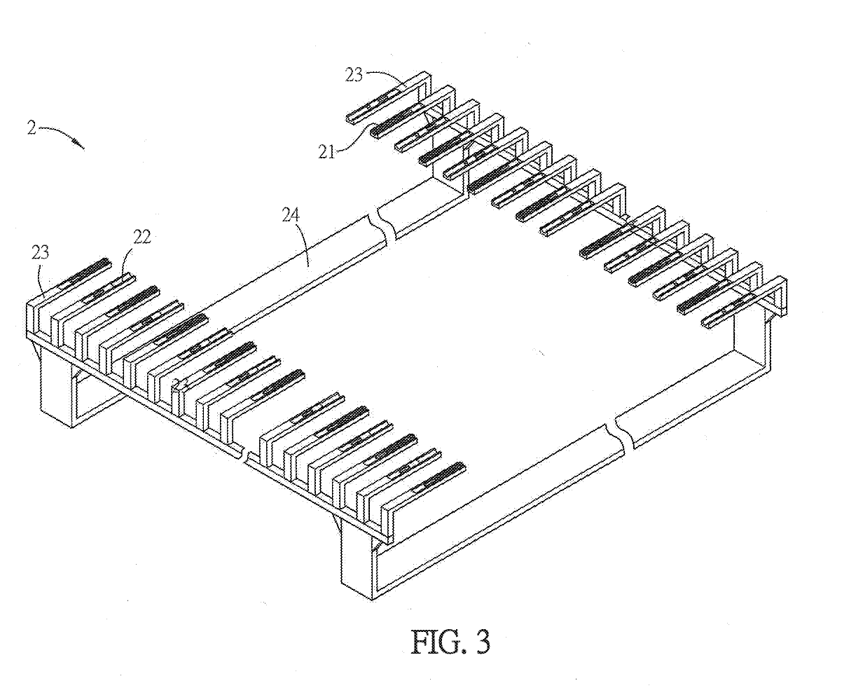

FIG. 3 is a schematic diagram of a part of the gait analysis device of the running exercise apparatus in FIG. 1. As shown in FIGS. 1 to 3, in this embodiment, the gait analysis device 2 further includes a connecting unit 24, which is connected to the supporting units 23 and fixed to the main body 1. As an embodiment, the connecting unit 24 is a frame structure that can be disposed at the edge of the main body 1 of the running exercise apparatus R so that the supporting units 23 can be systematically installed and removed instead of being assembled individually, and therefore the infrared modules can be located with higher accuracy. However, the scope of the invention doesn't narrow down to the shape and structure of the connecting unit 24. In other embodiments, the connecting unit 24 can be composed of two elements which are connected to the supporting units 23 on the opposite sides of the rolling portion 11, respectively. In this case, when the said two elements are installed to the running exercise apparatus R, the installation can be implemented from the two sides individually, and therefore the connecting unit is capable of fitting the running exercise apparatuses of different sizes.

However, the invention is not limited thereto. In other embodiments, the gait analysis device can have no connecting unit or the like. In this case, the supporting units are individually fixed to the running exercise apparatus, and therefore the location and the number of the supporting units can be adjusted. Hence, the assembly and disassembly can be done with more flexibility.

By the above-mentioned disposition, the infrared emitting unit 21 and the infrared receiving unit 22 of each of the infrared modules can be regarded as an emitter and a receiver, respectively, and the gait information can be detected and acquired by intercepting the communication between the emitter and receiver by the user's feet. The gait analysis by using the infrared module will be illustrated as below in detail.

As shown in FIG. 2, in this embodiment, the infrared emitting units 21 and the infrared receiving units 22 are disposed in a staggered arrangement, and that means the infrared emitting units 21 of the adjacent infrared modules are disposed on the opposite sides of the rolling portion 11, and likewise, the infrared receiving units 22 of the adjacent infrared modules are disposed on the opposite sides of the rolling portion 11. Accordingly, in comparison with the conventional art where the fewer infrared modules are disposed to reduce the interference, the interval between the infrared receiving units 22 of the adjacent infrared modules of this embodiment is increased so that the interference between the adjacent infrared modules can be avoided, and therefore the infrared modules can be disposed with a larger number so as to acquire the sufficient and more accurate gait information to obtain the more meaningful analysis result.

For simplifying the calculation and analysis about the signal received by the infrared receiving units 22, the intervals D between the adjacent infrared modules are kept uniform substantially. Through the above-mentioned disposition, the two sides of the rolling portion 11 can be equipped with the evenly distributed infrared modules. However, the above-mentioned disposition of the units is not for limiting the scope of the invention, and in the practical applications the interval between the adjacent infrared modules may contain some allowable error.

According to the size of the running exercise apparatus, the interval between the infrared modules and the interval between the units can be adjusted. For example, when the interval D between the adjacent infrared modules is 3 cm, the distance H (emitting distance) between the infrared emitting unit 21 and receiving unit 22 of each of the infrared modules is 55 cm, but the invention is not limited thereto. The interval D between the adjacent infrared modules can be 3 cm˜5 cm, and the distance H can be 55 cm˜80 cm, for example. The said interval or distance can be determined according to the applied running exercise apparatus.

The infrared emitting units 21 and the infrared receiving units 22 are disposed in a staggered arrangement, so the running exercise apparatus R having the gait analysis device 2 is more suitable for the usage of the unmodulated IR signal technique instead of the usage of the modulated IR signal technique which requires the higher cost and advanced technology. Hence, the running exercise apparatus R of the invention is further advantageous to reducing the cost and simplifying the production technology. However, the running exercise apparatus R of other embodiments also can cooperate with the modulated IR signal technique. That is to say, the infrared emitting unit 21 and the infrared receiving unit 22 can be the modulated type, and the interference can be further eliminated accordingly. The adoption of the modulated or unmodulated IR signal technique and the related details can be comprehended by those skilled in the art, and therefore they are not described here for conciseness.

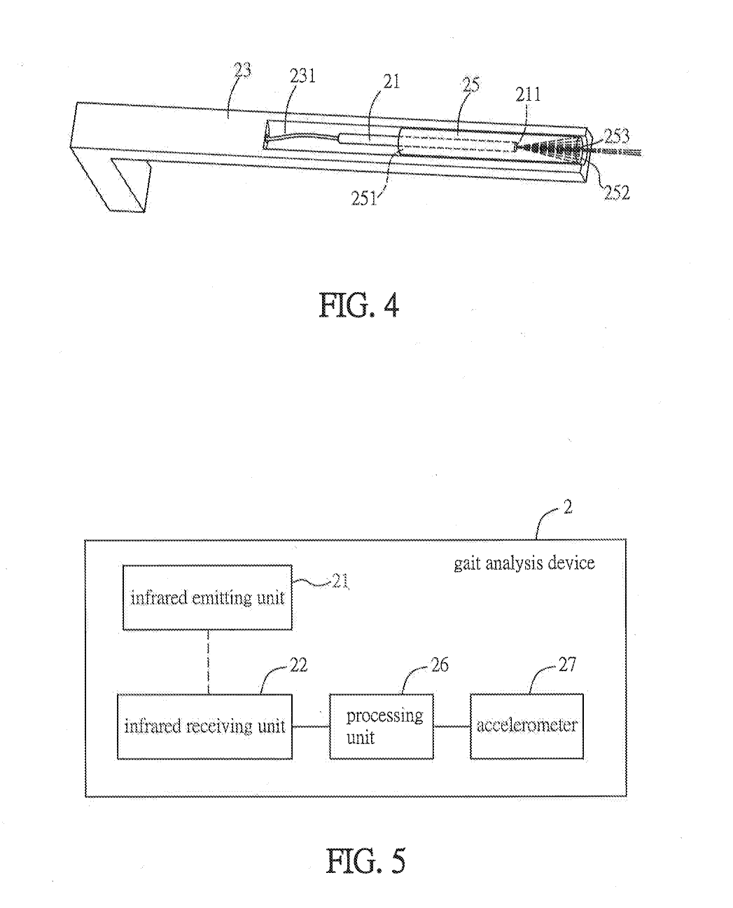

FIG. 4 is a schematic enlarged diagram of a part of the running exercise apparatus in FIG. 1. As shown in FIGS. 2 and 4, an end of each of the infrared emitting units 21 is covered by an opaque element 25 for further enhancing the detection effect of the infrared module. Herein for example, the opaque element 25 is a black and opaque tube. An end surface 251 of the opaque element 25 covers an emission end 211 of the infrared emitting unit 21 so that at least a part of the infrared emitting unit 21 is disposed within the opaque element 25 and not exposed. The other end surface 252 of the opaque element 25 has a hole 253, and the infrared emitting unit 21 can emit the infrared light through the hole 253 for the subsequent detection. Through the disposition of the opaque element 25, the scattering of the infrared light can be reduced and the interference can be thus avoided.

In the case where the scattering of the infrared emitted by the infrared emitting unit 21 is reduced, the aperture size of the hole 253 can be designed according to the size of the infrared emitting unit 21 for keeping a certain amount of the light through the hole 253. For example, the outer diameter of the infrared emitting unit 21 and the diameter of the hole 253 can be designed as the ratio of 1:1, but the invention is not limited thereto. Generally, when the diameter of the hole 253 is smaller, the anti-interference effect is better.

Besides, when the length of the opaque element 25 is longer, the covered range of the infrared emitting unit 21 is larger so that the scattering of the infrared light can be effectively avoided.

FIG. 5 is a schematic block diagram of the running exercise apparatus in FIG. 1. As shown in FIG. 5, the running exercise apparatus R further includes a processing unit 26, which is coupled to the infrared receiving units 22 to process the light signals received by the infrared receiving units 22. The signals outputted by the infrared modules and the analysis of the processing unit 26 about the signals will be illustrated in the following embodiment.

A gait analysis device is also disclosed in the invention. Since the technical features of the gait analysis device can be comprehended by referring to the above-mentioned running exercise apparatus R and the following embodiment, they are not described here for conciseness.

An embodiment as below is used to illustrate the gait analysis device applied to the running exercise apparatus and the gait analysis. To be noted, the following illustration is for the implementation for those skilled in the art, but is not for limiting the scope of the invention.

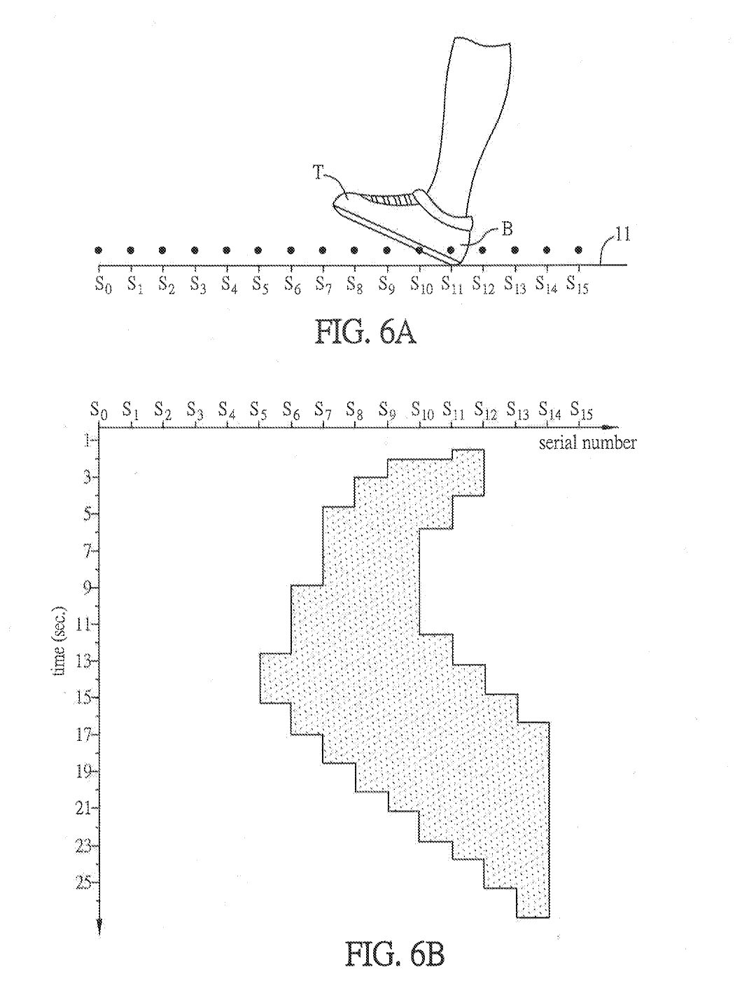

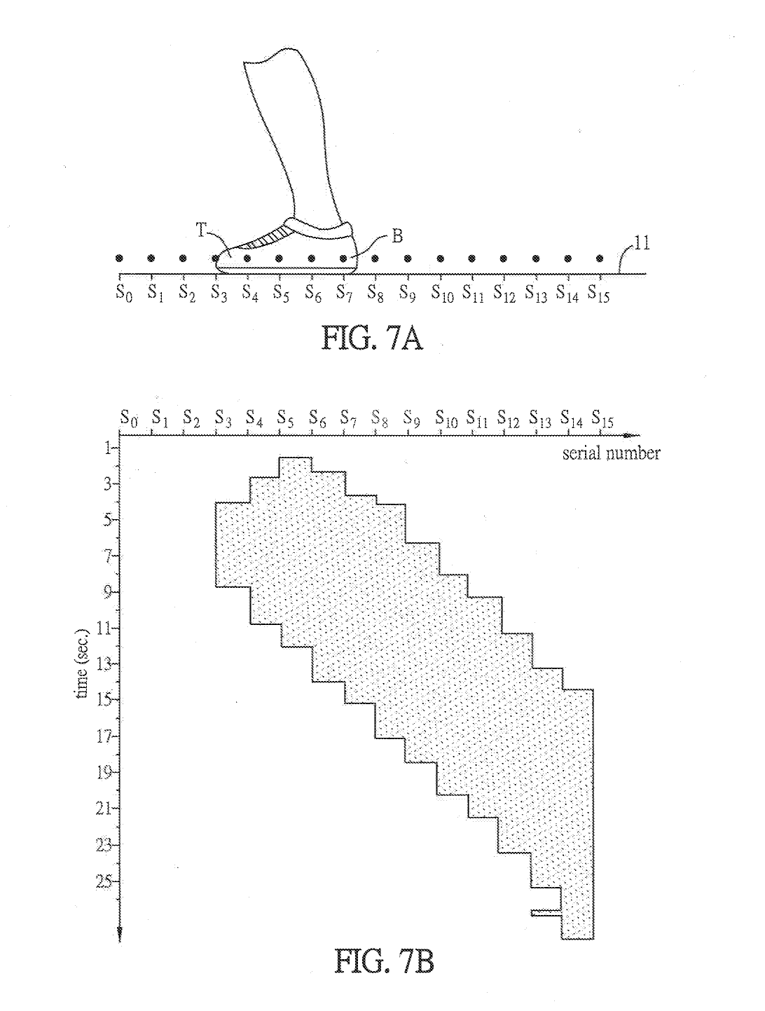

In this embodiment, a running exercise apparatus is used to obtain the gait information of a user, and the gait information includes the stride frequency, step length and landing strategy for example. The running exercise apparatus here is the running exercise apparatus R of the above embodiment, and therefore FIGS. 1 to 4 can be referred to for a further understanding. To facilitate the understanding, a plurality of the infrared modules of the gait analysis device 2 of the running exercise apparatus R parallel disposed on the two sides of the main body 1 are given a serial number (the serial number as S0˜S15 starts from the side near the display portion 12 as shown in FIGS. 6A and 7A). Accordingly, the user can stand on the rolling portion 11 and the feet of the user are driven to move by the rolling portion 11.

Since the infrared module applied in the invention belongs to the light-intercepting type, the light signal emitted by the infrared emitting unit 21 will be intermittently intercepted by the feet of the user when the user's feet move on the rolling portion 11. In this situation, the infrared receiving unit 22 will receive the intermittent light signals emitted by the infrared emitting unit 21, and the processing unit receiving the intermittent light signals can effectively analyze the timing, frequency or location of the interception of the light signal intercepted by the feet.

For the detection of the stride frequency, a single foot is set as a target, so the interval between two consecutive interceptions of a certain infrared module can be regarded as the period of the landing of the single foot. The stride frequency can be obtained by the reciprocal of the said period, and the average stride frequency can be obtained by averaging the stride frequencies obtain during a portion of time. The analysis of the periodic information and the calculation of the period and frequency can be comprehended by those skilled in the art, so they are not described here for conciseness.

For the detection of the step length, it needs to be implemented on condition that the user runs at a uniform running speed. As an embodiment, when the above stride frequency data has been acquired, the information of the step length can be obtained by the ratio of the running speed to the stride frequency. Likewise, the above calculation can be comprehended by those skilled in the art and is therefore not described here for conciseness.

In the gait analysis, the analysis data most related to the sports injuries, human health and even rehabilitation is the landing strategy. The feet landing of the user can be effectively obtained by the analysis result of the landing strategy, including, for example, landing by heels first or by toes first. The detection of the landing strategy will be clearly described in the following illustration, and since the measuring is implemented when the user wears shoes, the shoe heel B and the shoe toe T in FIGS. 6A and 7A are corresponding to the foot heel and the foot toe, respectively.

In this embodiment, the infrared module nearer the display portion 12 has a smaller serial number, so when the user lands with the shoe heel B first (as shown in FIG. 6A), the infrared module with a larger serial number is intercepted earlier than that with a smaller serial number (as shown in FIG. 6B) because the shoe heel B touches the rolling portion 11 earlier than the shoe toe T. In other words, when the shoe heel B lands first, the interception markers (serial number) will be distributed from rear to front as shown in FIG. 6B (S12 to S5) and then distributed from front to rear with the rolling of the rolling portion 11 (S5 to S12). On the contrary, when the shoe toe T lands first (as shown in FIG. 7A), the infrared module with a smaller serial number is intercepted earlier than that with a larger serial number (as shown in FIG. 7B), which indicates the interception markers (serial number) will be distributed from front to rear as shown in FIG. 7B (S3 to S15). Hence, by the interception sequence received by the infrared receiving units 22, the two kinds of the landing strategy can be determined and distinguished and the sports habit of the user can be improved thereby. More favorably, when the user undergoes foot rehabilitation, the landing strategy can be used to point out the muscle or joint to be concerned in order to provide better rehabilitation effect.

Furthermore, for determining the landing strategy more accurately, the gait analysis device 2 of the running exercise apparatus R of this embodiment further includes an accelerometer 27, which is coupled to the processing unit 26 (as shown in FIG. 5) and used to measure the accelerations along the triaxial directions (X, Y, Z) to output the signals accordingly. During the motion of the user, the accelerometer 27 can detect the acceleration at the instant of the landing of the user's foot on Z axis to generate an extremely large peak value (i.e. a strong signal on Z axis), and the accurate landing timing of the foot can be determined thereby. Favorably, because the processing unit 26 simultaneously receives the signals from the infrared receiving unit 22 and the accelerometer 27, the erroneous signal situation, which is caused when the foot swing accidentally intercepts the infrared emitting unit 21 because of the lower location of the infrared module, can be avoided. In other words, the accelerometer 27 is capable of effectively filtering out the erroneous signal caused during the foot swing. However, the accelerometer 27 can be disposed anywhere on the running exercise apparatus R as long as it doesn't affect the user's motion.

To be noted, the above-mentioned items of the gait analysis are just for example but not for limiting the scope of the invention. By the conversion formula or other analysis devices, the gait analysis apparatus 2 can acquire and analyze other required gait information.

In summary, the gait analysis device and the running exercise apparatus according to the invention have the infrared modules disposed on two sides of the running exercise apparatus, and the infrared emitting units and the infrared receiving units of the adjacent infrared modules are disposed in a staggered arrangement. Accordingly, the interval between the adjacent infrared receiving units can be increased and the interference therebetween can be thus effectively decreased. Besides, the emission end of the infrared emitting unit can be covered by an opaque element. The opaque element reduces the scattering of the infrared light emitted by the infrared emitting unit, and therefore the accuracy of the light signal obtained by the gait analysis device and the correctness of the analysis result can be both enhanced. Furthermore, the user can know the gait information of the motion thereby.

In order to avoid the interference between the infrared modules, the running exercise apparatus of the conventional art needs to use the modulated IR signal technique that requires the higher cost and higher level technology. However, in the invention, since the units are disposed in a staggered arrangement and the infrared emitting unit is covered by an opaque element, the gait analysis device can use the unmodulated IR signal technique but still reduce the interference caused by the unmodulated IR signal technique. Therefore, the invention is capable of reducing the cost of the running exercise apparatus.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A gait analysis device cooperated with a running exercise apparatus, comprising:
a plurality of infrared emitting units, wherein an end of each of the infrared emitting units is covered by an opaque element;
a plurality of infrared receiving units, wherein the infrared receiving units and the infrared emitting units are disposed in a staggered arrangement;
a plurality of supporting units, wherein the infrared receiving units and the infrared emitting units are disposed to the supporting units; and
a processing unit coupled with the infrared receiving units.

2. The gait analysis device as recited in claim 1, wherein each of the infrared emitting units, each of the infrared receiving units or each of the supporting units has a bent angle corresponding to at least an edge of the running exercise apparatus.

3. The gait analysis device as recited in claim 1, further comprising:
a connecting unit connected to the supporting units.

4. The gait analysis device as recited in claim 1, wherein the infrared emitting units are modulated IR emitting units and the infrared receiving units are modulated IR receiving units.

5. The gait analysis device as recited in claim 1, wherein one of the infrared emitting units and the infrared receiving unit adjacent to the infrared emitting unit have an interval of 3 cm˜5 cm.

6. The gait analysis device as recited in claim 1, wherein the distance between one of the infrared emitting units and the infrared receiving unit that is opposite to the infrared emitting unit is 55 cm˜80 cm.

7. The gait analysis device as recited in claim 1, further comprising:
an accelerometer coupled to the processing unit.

8. A running exercise apparatus, comprising:
a main body; and
a gait analysis device disposed corresponding to the main body and comprising:
a plurality of infrared emitting units, wherein an end of each of the infrared emitting units is covered by an opaque element;
a plurality of infrared receiving units, wherein the infrared receiving units and the infrared emitting units are disposed in a staggered arrangement;
a plurality of supporting units, wherein the infrared receiving units and the infrared emitting units are disposed to the supporting units; and
a processing unit coupled with the infrared receiving units.

9. The running exercise apparatus as recited in claim 8, wherein each of the infrared emitting units, each of the infrared receiving units or each of the supporting units has a bent angle corresponding to at least an edge of the main body.

10. The running exercise apparatus as recited in claim 8, further comprising:
a connecting unit connected to the supporting units.

11. The running exercise apparatus as recited in claim 8, wherein the infrared emitting units are modulated IR emitting units and the infrared receiving units are modulated IR receiving units.

12. The running exercise apparatus as recited in claim 8, wherein one of the infrared emitting units and the infrared receiving unit adjacent to the infrared emitting unit have an interval of 3 cm˜5 cm.

13. The running exercise apparatus as recited in claim 8, wherein the distance between one of the infrared emitting units and the infrared receiving unit that is opposite to the infrared emitting unit is 55 cm˜80 cm.

14. The running exercise apparatus as recited in claim 8, wherein the gait analysis device further comprises:
an accelerometer coupled to the processing unit.
